(19) United States
(12) Patent Application Publication
(10) Pub. No.: US 2024/0277469 A1
(43) Pub. Date: Aug. 22, 2024

(54) Methods and Devices for Treating Tricuspid Valve Disease

(71) Applicants: Vaikom S. Mahadevan, Westborough, MA (US); Michael J. Horzewski, San Jose, CA (US)

(72) Inventors: Vaikom S. Mahadevan, Westborough, MA (US); Michael J. Horzewski, San Jose, CA (US)

(21) Appl. No.: 18/583,369

(22) Filed: Feb. 21, 2024

Related U.S. Application Data
(60) Provisional application No. 63/447,614, filed on Feb. 22, 2023.

(51) Int. Cl. A61F 2/24; A61F 2/06

(52) U.S. Cl. CPC A61F 2/2418; A61F 2/06; A61F 2/2436; A61F 2220/0008

(57) ABSTRACT

Systems and methods for treatment of tricuspid valve disease. The systems and methods can anchor a supplemental valve apparatus in the vena cavae and provide a competent valve positioned in the right atrium or in or through the tricuspid annulus without requiring an anchor in the tricuspid annulus. The supplemental valve apparatus can include a supplemental valve coupled to at least one supplemental valve structure, which can extend into at least one of the inferior vena cava or superior vena cava with the supplemental valve positioned in or adjacent the right atrium. The valve apparatus can be delivered percutaneously by a delivery system, which can include the supplement valve apparatus positioned between an inner and outer shaft that can be retracted to deploy the supplemental valve apparatus in or adjacent the right atrium. Methods of delivery and treating a tricuspid valve by a supplement valve apparatus are also described herein.

### CROSS-REFERENCES TO RELATED APPLICATIONS

The present application claims the benefit under 35 USC § 119(e) of U.S. Provisional Appln. No. 63/447,614 filed Feb. 22, 2023; the full disclosure which is incorporated herein by reference in its entirety for all purposes.

### BRIEF SUMMARY OF THE INVENTION

Technical Field Described are systems and methods for the treatment of tricuspid valve disease. The systems and methods can be used in which a new supplemental valve apparatus that anchors in the vena cavae and is able to provide a competent valve positioned in the right atrium or in or through the tricuspid annulus without needing an anchor in the tricuspid annulus.

### BACKGROUND OF THE INVENTION

For patients with tricuspid valve disease, there is increasingly recognized clinical benefit to treat severe tricuspid regurgitation. Traditionally, management of tricuspid valve disorders has been accorded lesser importance than that of left-sided valvular heart disease and the tricuspid valve has often been alluded to as the “forgotten valve”. Recently, increased appreciation of the long-term adverse consequences of tricuspid valve disease, and in particular of severe tricuspid regurgitation, coupled with continued advances in surgical and percutaneous transcatheter techniques, has led to more aggressive treatment recommendations.

There is widespread recognition, however, that the evidence base underlying current guideline recommendations for management of patients with tricuspid valve disease does not include randomized controlled trials to inform clinical practice. This is primarily because of the lack of any reliable device therapy for tricuspid regurgitation. The vast majority of tricuspid regurgitation is caused by annular dilation due to various causes including atrial fibrillation which is a common clinical problem with advancing age.

The Triclip was studied in the Tricuspid position (TRILUMINATE trial) and has just been approved by the FDA for clinical use. There is also a similar trial by the Pascale device. The NAVIGATE valve is an annulus-based device and has been studied for compassionate use in and a trial is being planned. The Tric valve which is essentially a valve in the superior vena cava and inferior vena cava has been studied. The EVOQUE valve was recently approved based on the TRISCEND trial is based on implantation in the tricuspid annulus. If comes in three sizes and is delivered using a large 28 Fr delivery system.

There is an opportunity for a new valve for the treatment of tricuspid valve disease, which anchors in the vena cavae and is able to provide a competent supplemental valve positioned in the right atrium or the tricuspid annulus without needing an anchor in the annulus since the expansile nature of the annulus and dilatation makes such anchoring very difficult. The supplemental valve apparatus is designed on this concept, is self-expanding, and can be retrievable and repositionable. The supplemental valve apparatus overcomes the issues with native tricuspid valve replacement, due to a) the native tricuspid valve annulus is soft, pliable, and it dilates, providing insufficient structural strength to properly anchor a replacement native tricuspid valve, b) it eliminates the need to achieve a leak-free seal at the native tricuspid valve annulus by making the seal in the superior vena cava and inferior vena cava, and c) the functionality and seal of the supplemental valve apparatus is not impacted by degeneration of the right atrium and native tricuspid valve annulus over time (as the patient ages).

In addition, the supplemental valve apparatus may be used even if a replacement tricuspid valve has been implanted, such as when the functionality of a replacement tricuspid valve is compromised or following failure of repair of the tricuspid valve using transcatheter therapies such as the Triclip.

### BRIEF SUMMARY OF THE INVENTION

In accordance with one aspect of the present technology, there is provided a system useable for implanting a supplemental valve apparatus within the body of a human or animal subject, such system comprising a supplemental valve apparatus and a delivery system.

The system includes a supplemental valve apparatus which enables the management of blood flow without replacing the native tricuspid valve or replacement tricuspid valve. The supplemental valve apparatus operates as a fully-functioning tricuspid valve while the native or replacement, compromised tricuspid valve is left in place.

Still further in accordance with the present technology, the supplemental valve apparatus does not anchor in the native tricuspid valve annulus. The supplemental valve apparatus is held in position by elements (legs) located and/or anchored in the superior vena cava and in the inferior vena cava.

Still further in accordance with the present technology, the supplemental valve component of the supplemental valve apparatus may be located within the right atrium and sufficiently spaced from the native tricuspid valve or replacement tricuspid valve as to not impact the function of the supplemental valve.

Still further in accordance with the present technology, the supplemental valve may be located within the right atrium and positioned into or through the native tricuspid valve to accommodate tricuspid valve stenosis and hold open the native tricuspid valve.

Still further in accordance with the present technology, the supplemental valve may have one or more leaflets, or a ball and cage configuration to enable management of blood flow through the supplemental valve.

Still further in accordance with the present technology, there is provided a jacket (e.g., graft or graft-like material) used to manage blood flow and direct it from the superior vena cava and inferior vena cava through the supplemental valve.

Still further in accordance with the present technology, the valve leg of the supplemental valve apparatus is able to be deployed and its orientation optimized before deployment of either the superior vena cava or inferior vena cava legs.

Still further in accordance with the present technology, the supplemental valve apparatus is self-expanding and is recapturable prior to the final leg being fully deployed. This enables the ability to test the supplemental valve and the seal of the first-deployed anchoring leg prior to complete deployment of the supplemental valve apparatus.

Still further in accordance with the present technology, there is provided a supplemental valve apparatus and delivery system which is delivered from an inferior access location (e.g., femoral vein) to the deployment site. The delivery system enables the superior vena cava leg and supplemental valve to be deployed prior to the inferior vena cava leg, such that the supplemental valve and superior vena cava leg seal can be tested with blood flow from the head prior to fully deploying the inferior vena cava leg.

Still further in accordance with the present technology, there is provided a supplemental valve apparatus and delivery system which is delivered from an inferior access location to the deployment site. The delivery system enables the inferior vena cava leg to be deployed prior to fully deploying the superior vena cava leg which allows for very accurate placement of the inferior vena cava leg in relation to the hepatic veins so as to not occlude blood flow to the hepatic veins yet provide sufficient anchoring length for the inferior vena cava leg. As the inferior vena cava leg may be shorter than the superior vena cava leg, this also allows for any overall length difference between the supplemental valve inferior vena cava to superior vena cava length and the length of the native vessels/right atrium.

Still further in accordance with the present technology, there is provided a supplemental valve apparatus and delivery system which is delivered from a superior access location (e.g., jugular vein). Delivering the supplemental valve apparatus from a superior location allows for very accurate placement of the inferior vena cava leg in relation to the hepatic veins so as to not occlude blood flow to the hepatic veins yet provide sufficient anchoring length for the inferior vena cava leg. As the inferior vena cava leg may be shorter than the superior vena cava leg, this also allows for any overall length difference between the supplemental valve inferior vena cava to superior vena cava length and the length of the native vessels/right atrium.

Still further in accordance with the present technology, there is provided a supplemental valve apparatus and delivery system which is delivered from a superior access location (e.g., jugular vein). The delivery system enables the superior vena cava leg and supplemental valve to be deployed prior to the inferior vena cava leg, such that the supplemental valve and superior vena cava leg seal can be tested with blood flow from the head prior to fully deploying the inferior vena cava leg.

Still further in accordance with the present technology, the inferior vena cava leg of the supplemental valve apparatus can have a length of unwrapped section to extend the anchoring portion across at least part of the hepatic veins to further increase anchoring robustness. In addition, the superior vena cava leg may also have a similar unwrapped section.

Still further in accordance with the present technology, one or both of the superior vena cava and inferior vena cava legs of the supplemental valve apparatus may be formed with an increased diameter, protrusion(s), or cuff to enhance the seal of one or both of these legs.

Still further in accordance with the present technology, the supplemental valve apparatus may include a skirt adjacent the valve leg which can be used to isolate (seal off) a portion of the native right atrium. The skirt may extend beyond or behind the plane of the supplemental valve to isolate the desired amount of native right atrium.

Still further in accordance with the present technology, is a handle for deploying the supplemental valve apparatus from the superior vena cava leg towards the inferior vena cava leg of the supplemental valve apparatus.

Still further in accordance with the present technology is a handle for deploying the supplemental valve apparatus from the inferior vena cava leg towards the superior vena cava leg of the supplemental valve apparatus.

Still further in accordance with the present technology is a handle for deploying the supplemental valve apparatus, with the supplemental valve leg being deployed first, followed by the inferior vena cava leg and/or the superior vena cava leg being deployed second, or in incremental steps, or at the same time.

### BRIEF DESCRIPTION OF THE DRAWINGS

A general architecture that implements the various features of the present technology will now be described with reference to the drawings. The drawings and the associated descriptions are provided to illustrate some but not all embodiments or examples of the present technology and do not limit the scope of the claimed inventions in any way. Throughout the drawings, reference numbers are re-used to indicate correspondence between referenced elements.

FIG. 1 illustrates an embodiment of the supplemental valve apparatus.

FIGS. 2A-B illustrate embodiments of the supplemental valve apparatus with the jacket removed.

FIG. 3 illustrates an embodiment of the supplemental valve apparatus with the supplemental valve positioned through the native tricuspid valve.

FIG. 4 illustrates an embodiment of the supplemental valve apparatus used when a replacement tricuspid valve is present in the patient.

FIG. 5 illustrates an embodiment of a delivery system.

FIG. 6 illustrates an embodiment of a portion of a delivery system inner shaft.

FIG. 7 illustrates an additional embodiment of a portion of a delivery system inner shaft.

FIG. 8 illustrates an embodiment of a delivery system outer shaft.

FIG. 9 illustrates an embodiment of the distal portion of a delivery system with the supplemental valve apparatus constrained within the delivery system.

FIG. 10A illustrates an embodiment of a handle in a position with the supplemental valve apparatus is constrained within the delivery system of FIG. 9.

FIG. 10B illustrates an embodiment of a handle in a position where the supplemental valve apparatus has been released from the delivery system of FIG. 9.

FIG. 11 illustrates an additional embodiment of the distal portion of a delivery system with the supplemental valve apparatus constrained within the delivery system.

FIG. 12A illustrates an embodiment of a handle in a position with the supplemental valve apparatus is constrained within the delivery system of FIG. 11.

FIG. 12B illustrates an embodiment of a handle in a position where the supplemental valve apparatus has been released from the delivery system of FIG. 11.

FIG. 13 illustrates an additional embodiment of the distal portion of a delivery system with the supplemental valve apparatus constrained within the delivery system.

FIG. 14 illustrates an additional embodiment of the distal portion of a delivery system with the supplemental valve apparatus partially deployed from the delivery system.

FIG. 15A illustrates an embodiment of a handle in a position with the supplemental valve apparatus is constrained within the delivery system of FIGS. 13-14.

FIG. 15B illustrates an embodiment of a handle in a position where the supplemental valve apparatus has been released from the delivery system of FIGS. 13-14.

### DETAILED DESCRIPTION OF THE INVENTION

The technology disclosed herein may be embodied in other specific forms without departing from its spirit or essential characteristics. The described embodiments are to be considered in all respects only as illustrative and not restrictive. The scope of the technology is therefore indicated by the appended claims rather than the foregoing description. All changes that come within the meaning and range of equivalency of the claims are to be embraced within their scope.

The supplemental valve apparatus 10 and a delivery system 1000 are used as part of a procedure to place a competent valve in the right atrium 20 or into or through the native tricuspid valve 30 annulus.

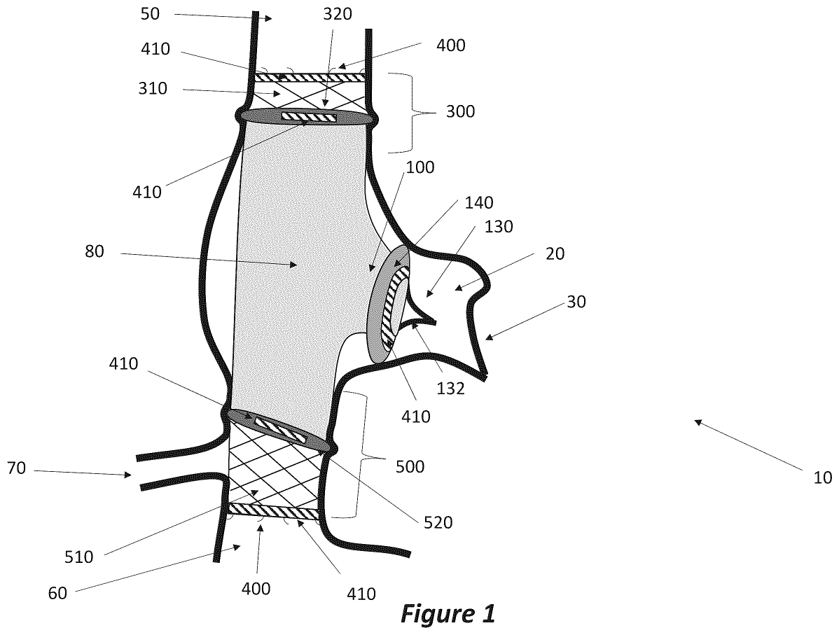

FIG. 1 illustrates an embodiment of the supplemental valve apparatus 10. The supplemental valve apparatus 10 enables the management of blood flow without replacing the native tricuspid valve 30 or having to replace a replacement tricuspid valve 40, if present. The supplemental valve apparatus 100 operates as a fully-functioning tricuspid valve while the native or replacement, compromised tricuspid valve is left in place. The supplemental valve 130 may have, for example, one or more leaflets 132, a ball and cage configuration, or any configuration that serves to function as a valve. The supplemental valve 130 may typically have 2 or 3 leaflets 132. The supplemental valve apparatus 10 does not require an anchor 400 in the native tricuspid valve 30 annulus, but may have one or more anchors 400 located in the native tricuspid valve 30 if desired. The supplemental valve apparatus 10 is entirely or in part held in position by elements (legs) in the superior vena cava 50 and in the inferior vena cava 60.

The supplemental valve apparatus 10 has up to three legs, the supplemental valve leg 100, the superior vena cava leg 300, and the inferior vena cava leg 500. One or more structures form the body of the supplemental valve apparatus 10, which may include a single underlying structure, such as a metallic structure, to enable the superior vena cava leg 300 to be positioned in the superior vena cava 50, and the inferior vena cava leg 500 to be positioned in the inferior vena cava 60, and support positioning of the supplemental valve leg 100 in the right atrium 20. The body structure may be at least partially covered, inside and/or outside, by a jacket 80. The jacket 80 is intended to direct blood flow from the superior vena cava 50 and inferior vena cava 60 into and through the supplemental valve 130. As the supplemental valve apparatus 10 is located on the venous side and therefore lower pressure than the arterial side, the jacket 80 can be made from a relatively thin material. The jacket 80 can be made of PTFE, typical graft materials, or other polymers or tissues and may be flexible to accommodate delivery and offer some level of compliance, so long as the jacket 80 directs blood flow through the supplemental valve 130.

The supplemental valve leg 100 supports the supplemental valve 130. The supplemental valve 130 can be positioned in the right atrium 20, or within or through the native tricuspid valve 30. Typical placement within the right atrium 20 would be from 0.5 to 4.0 cm, and more specifically 1.0 cm to 3.0 cm into the right atrium 20. The supplemental valve 130 can be a biologic, such as a cow or pig valve, or a synthetic material, metal, or a combination thereof, typically having one or more leaflets 132, a ball and cage configuration, etc. and may include a supplemental valve support 120 structure which may be made from one or more of a biologic, polymer, or a metal, such as NiTi (NiTi as used herein includes NiTi and NiTi alloys). Typical size range for the diameter of the supplemental valve is 25 mm to 55 mm, and may be provided in a variety of sizes to fit the patient, such as 32 mm, 38 mm, 45 mm, and 50 mm. The supplemental valve 130 is coupled to the supplemental valve leg 100. Attachment can be by bonding, welding (e.g., metal supplemental valve leg struts 110 welded to a metal supplemental valve support 120 structure), sewing, or other attachment mechanisms.

The supplemental valve 130 and/or supplemental valve leg 100, and/or jacket 80 may include a skirt 140. The skirt 140 can provide a seal from the jacket 80 and/or supplemental valve 130 to the right atrium 20 wall, at, in front of, and/or behind the plane of the supplemental valve 130. The skirt 140 can be made from a biologic and/or a polymer, such as from typical graft materials, or other material and can be used reduced the amount of blood volume of the right atrium 20, and also to reduce the propensity for stagnant blood within the right atrium 20, which could lead to thrombus. The skirt 140 may include a support structure, such as struts or a mesh (e.g., NiTi) and may also include attachment and/or anchoring elements to maintain the skirt 140 position/seal to the right atrium 20 wall.

Radiopaque elements 410 may be used to locate one or more components or locations of the supplemental valve apparatus 10, for example, the supplemental valve leg 100, supplemental valve support 120, superior vena cava leg 300, superior vena cava leg sealing cuff 320, inferior vena cava leg 500, inferior vena cava leg sealing cuff 520, anchors 400, etc. In certain embodiments, components themselves may be radiopaque, coated/filled with a radiopaque material, or contain radiopaque materials.

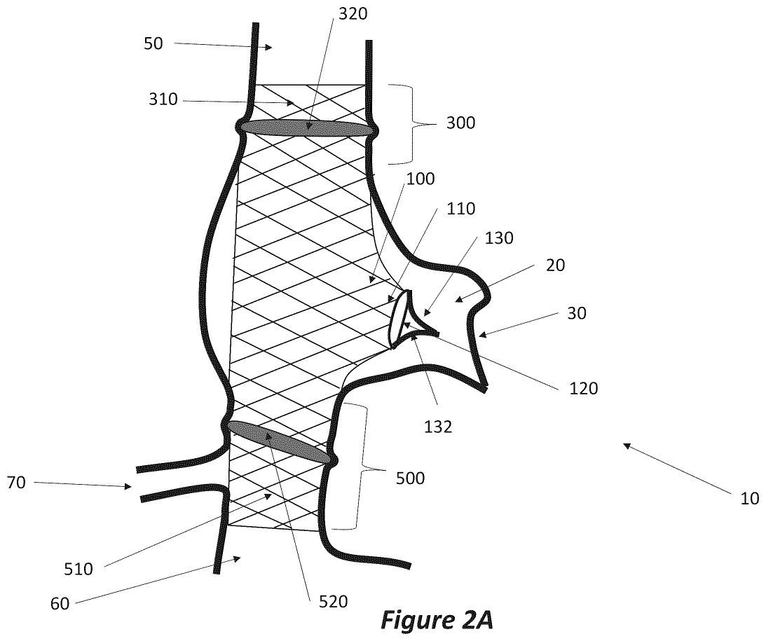

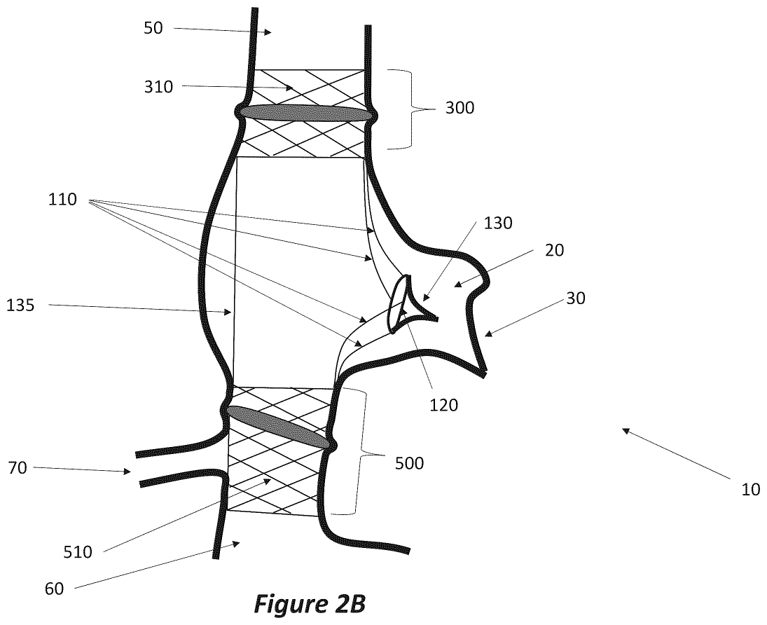

FIGS. 2A-B illustrate embodiments of the supplemental valve apparatus with the jacket 80 removed. The supplemental valve leg struts 110 are connected, either directly or indirectly, to either one of or both of the superior vena cava leg struts 310 and the inferior vena cava leg struts 510. One or more length struts 135 may be used to connect the superior vena cava leg struts 310 and/or the inferior vena cava leg struts 510 and/or the supplemental valve leg struts 110. The combination of the supplemental valve leg struts 110 and/or the superior vena cava leg struts 310 and/or the inferior vena cava leg struts 510 and/or length struts 135 form the body, which at least in part, holds the supplemental valve 130 in position. The skirt 140 and/or additional support structures (e.g., struts to or adjacent the native tricuspid valve 30) may be incorporated to provide positional locating elements. These struts can be formed from a mesh or stent-like structure as well as other configurations. The body may be for example, a mesh, a laser cut or etched material, a braided material, filaments/wires, or any combination of structures and manufacturing techniques to form a non-solid-walled structure.

The superior vena cava leg 300 is positioned/anchored at least in part within the superior vena cava 50. The superior vena cava leg 300 can extend up to 3 cm or more into the superior vena cava 50. The superior vena cava leg 300 may have anchors 400, such as hooks or barbs, to maintain position within the superior vena cava 50, and/or be matched or oversized in diameter compared to the native vessel by up 30% or more to provide for maintenance of the deployed position. Typical diameters for the superior vena cava leg 300 are 25 mm to 45 mm, more typically 30 mm to 40 mm. The jacket 80 may extend about the superior vena cava leg 300 into the superior vena cava 50, as long as the jacket 80 does not occlude the internal jugular vein. The superior vena cava leg struts 310 can be formed from a mesh or stent-like structure as well as other configurations. The body may be for example, a mesh, a laser cut or etched material, a braided material, or any combination of structures and manufacturing techniques to form a non-solid-walled structure.

The superior vena cava leg 300 may include a superior vena cava sealing cuff 320, which can be a portion of the superior vena cava leg 500 that is enlarged in diameter. The jacket 80 may extend to (depicted in FIG. 1) or beyond the superior vena cava sealing cuff 320. The superior vena cava sealing cuff 320 may be formed integral with the jacket 80. The superior vena cava sealing cuff 320 may be a separate element coupled to or formed integral with the superior vena cava leg struts 310. The enlarged diameter may be from 1% to 30% or more than the native superior vena cava 50 diameter. The length of the superior vena cava sealing cuff 320 may be from 1 mm to 20 mm, more preferably from 1 mm to 5 mm in length.

The inferior vena cava leg 500 is positioned/anchored at least in part within the inferior vena cava 60. The inferior vena cava leg 500 can extend up to 3 cm or more into the inferior vena cava 60. The inferior vena cava leg 500 may have anchors 400, such as hooks or barbs, to maintain position within the inferior vena cava 60, and/or be matched or oversized in diameter compared to the native vessel by up 30% or more to provide for maintenance of the deployed position. Typical diameters for the inferior vena cava leg 500 are 25 mm to 45 mm, more typically 30 mm to 40 mm. The jacket 80 may extend about the inferior vena cava leg 500 into the inferior vena cava 60, as long as the jacket 80 does not occlude the hepatic vein. The inferior vena cava leg struts 510 can be formed from a mesh or stent-like structure as well as other configurations. The body may be for example, a mesh, a laser cut or etched material, a braided material, or any combination of structures and manufacturing techniques to form a non-solid-walled structure. The inferior vena cava leg 500 may extend across and beyond the hepatic vein to provide additional support. An open cell, mesh, or other configuration which does not extensively limit blood flow through it enables the inferior vena cava leg 500 to be positioned in this manner.

The inferior vena cava leg 500 may include an inferior vena cava sealing cuff 520, which can be a portion of the inferior vena cava leg 500 that is enlarged in diameter. The jacket 80 may extend to (depicted in FIG. 1) or beyond the inferior vena cava sealing cuff 520. The inferior vena cava sealing cuff 520 may be formed integral with the jacket 80. The inferior vena cava sealing cuff 520 may be a separate element coupled to or formed integral with the inferior vena cava leg struts 510. The enlarged diameter may be from 1% to 30% or more than the native inferior vena cava 60 diameter. The length of the inferior vena cava sealing cuff 520 may be from 1 mm to 20 mm, more preferably from 1 mm to 5 mm in length.

The inferior vena cava leg sealing cuff 520 may be titled with respect to the longitudinal axis of the inferior vena cava 60 depicted in FIG. 1. Tilting the inferior vena cava sealing cuff 520 enables the inferior vena cava leg sealing cuff 520 to extend further into the inferior vena cava 60 without occluding the hepatic vein, likely providing a better engagement of the inferior vena cava leg 500 within the inferior vena cava 60.

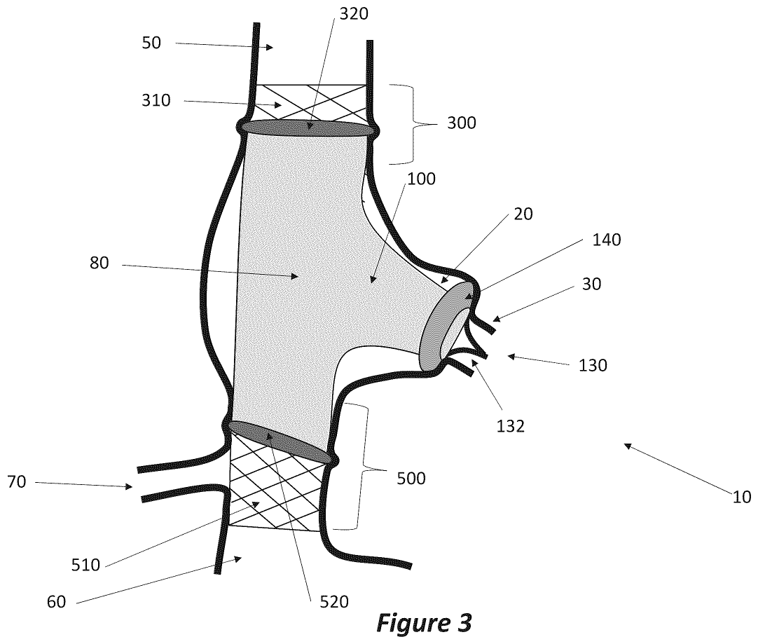

As depicted in FIG. 3, the supplemental valve 30 may be positioned adjacent or through the native tricuspid valve 30. This placement allows for the supplemental valve leg 100 to use the cardiac tissue for additional support and for location of the supplemental valve 130.

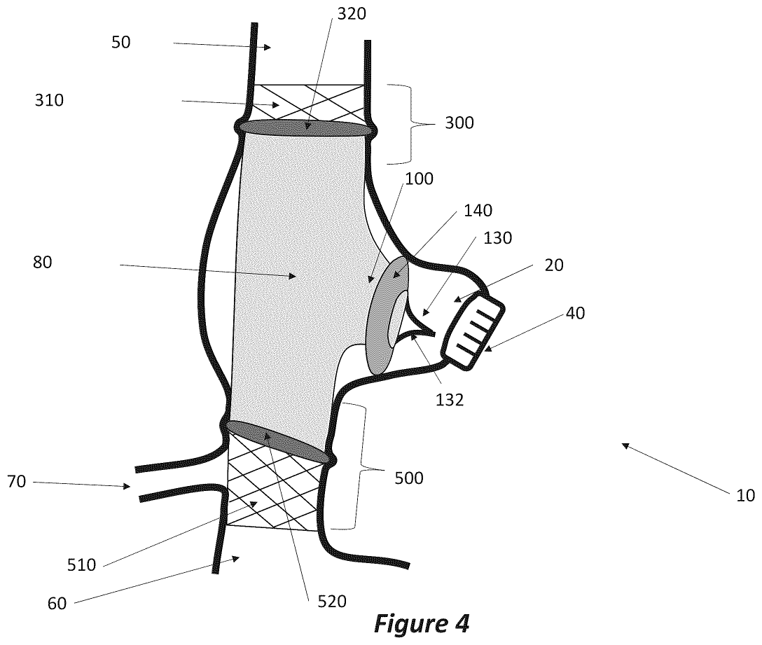

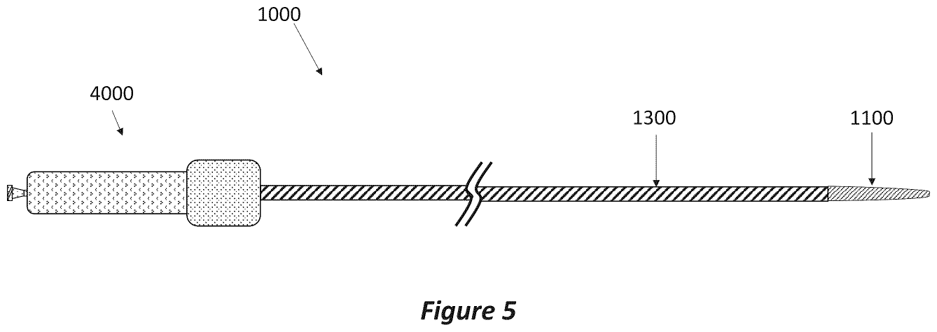

The design of the supplemental valve apparatus 10 enables the supplemental valve apparatus 10 to be placed within a patient that has an existing replacement tricuspid valve 40 already implanted (see FIG. 4). This is especially useful when the replacement tricuspid valve 40 has or is beginning to be compromised. In this scenario, it is preferable to have the supplemental valve 130 positioned within the right atrium 20, such that the supplemental valve 130 is not in direct contact with the replacement tricuspid valve 40.

In certain embodiments, the diameter sizing of the superior vena cava leg 300 and inferior vena cava leg 500 may be similar or staggered to properly match the patient's anatomy. For example, the superior vena cava leg 300 and inferior vena cava leg 500 may both be 30 mm, 35 mm, 40 mm, or 45 mm in diameter, while in other cases the superior vena cava leg 300 may be larger in diameter than the inferior vena cava leg 500, e.g., 40 mm vs. 35 mm or 35 mm vs. 30 mm, or the inferior vena cava leg 500 may be larger in diameter than the superior vena cava leg 300, e.g., 40 mm vs 35 mm or 35 mm vs. 30 mm. The distance between the superior vena cava leg sealing cuff 320 and the inferior vena cava leg sealing cuff 520 may typically be from 3 cm to 8 cm, and more commonly from 4.5 cm to 7 cm.

An example configuration for a supplemental valve apparatus 100 for use in the right atrium 20, anchored in the superior vena cava 50 and the inferior vena cava 60, the supplemental valve apparatus 10 having a supplemental valve leg 100 extending into the right atrium 20, the supplemental valve 130 containing a 32 mm diameter biologic valve, the existing tricuspid valve 30 (or replacement valve 40) are left in place (i.e., not removed from the patient). The supplemental valve 130 is sewn to the supplemental valve support 120, and positioned 2 cm into the right atrium on the supplemental valve leg 100. The supplemental valve leg 100 has multiple supplemental valve legs struts 110, coupled to a supplemental valve support 120, extending or coupled to superior vena cava leg struts 310 and inferior vena cava leg struts 510, and is made from NiTi. The struts support a non-thrombogenic jacket 80, made from Dacron (or PTFE), from the superior vena cava sealing cuff 320 to the inferior vena cava sealing cuff 520, and to the supplemental valve 130, to direct blood flow from the inferior vena cava 60 and superior vena cava 50 through the supplemental valve 130. The inferior vena cava leg 500 having an inferior vena cava leg sealing cuff 520 that is angled with respect to the longitudinal axis of the inferior vena cava 60 (as shown in FIGS. 1-4). The angle enabling the inferior vena cava leg sealing cuff 520 to not compromise flow from the hepatic vein 70. The inferior vena cava leg struts 510 extend beyond the hepatic vein 70 for support, but the jacket 80 does not. The superior vena cava leg 300 extends into the superior vena cava 50 with superior vena cava leg struts 310 extending beyond the superior vena cava leg sealing cuff 320. The sealing cuffs are also made from Dacron (or PTFE). The superior vena cava leg 300 has a diameter of 35 mm and the superior vena cava leg struts extend 15 mm into the superior vena cava 50. The inferior vena cava leg 500 has a diameter of 35 mm and the inferior vena cava leg struts 510 extend 10 mm into the inferior vena cava 60. The leg sealing cuffs are 5% greater in diameter than the leg diameters and 3 mm in length.

To deploy the supplemental valve apparatus 10, it is necessary to have a delivery system 1000. In certain embodiments, the delivery system 1000 is also able to recapture the supplemental valve apparatus 10 after partial deployment. The major components of the delivery system 1000 consist of the inner shaft 1010, outer shaft 1300, and handle 4000, 5000. The delivery system 1000 is preferably 30 F or less in diameter, more preferably 20 F or less in diameter.

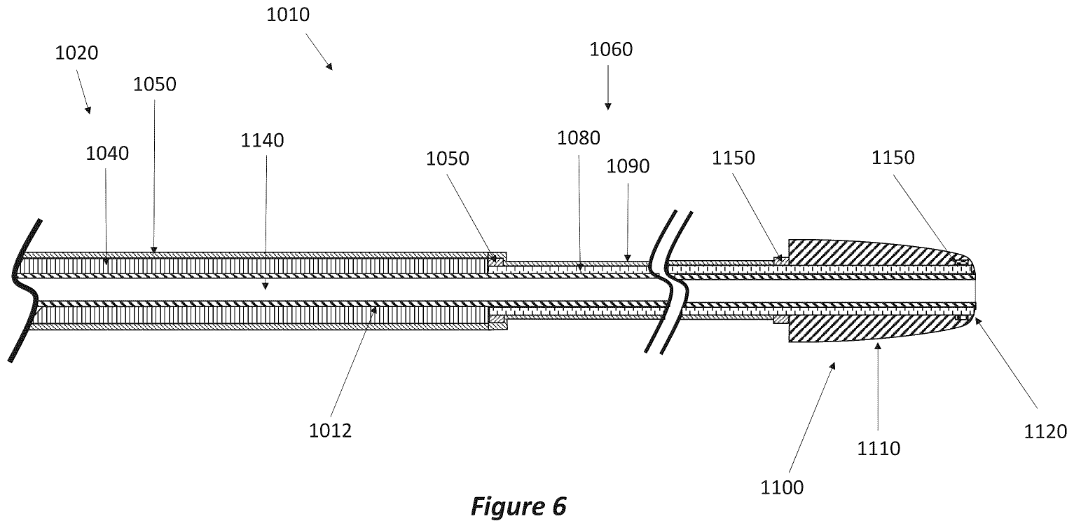

The inner shaft 1010 can be comprised of three or more regions (FIG. 6), the shaft proximal to where the supplemental valve apparatus 10 is positioned, the region including where the supplemental valve apparatus 10 is positioned, and the region distal to where the supplemental valve apparatus 10 is positioned. The shaft proximal to where the supplemental valve apparatus 10 is positioned is depicted in FIG. 6 as having one region, the proximal inner shaft body 1020 though the proximal inner shaft body 1020 can be constructed with more than one region to provide another approach to having variable stiffness along the length of the proximal inner shaft body 1020. The proximal inner shaft body 1020 can be made from a combination of one or more layers/components, including but not limited to a proximal inner shaft structural member 1040 and a proximal inner shaft outer member 1050, and, or any individual component or combination thereof, such as the proximal inner shaft body 1020 being made from a PTFE impregnated polyimide with or without a proximal inner shaft outer member 1050. The proximal inner shaft structural member 1040 generally provides column strength to the proximal inner shaft body 1020.

The proximal inner shaft structural member 1040 can be made from, for example, one or more of the following: polymer, metal, polyimide, polyethylene, polyurethanes, polyamides and blends (e.g., Pebax®), stainless steel, NiTi, braided, coils, thermosets and thermoplastics.

The outer surface of the proximal inner shaft outer member 1050 is preferably lubricious/low-friction (e.g., high density polyethylene, PTFE, etc.) with respect to the inner surface of the outer shaft 1300 over the range of motion of the outer shaft 1300 with respect to the inner shaft 1010. This aides in the outer shaft 1300 being easily retracted with respect to the inner shaft 1010 during supplemental valve apparatus 10 deployment.

In any of the embodiments described herein, it may be desirable to change the flexibility of the inner shaft 1010 along the length. This may be accomplished by changing thickness or properties of materials, including the proximal inner shaft structural member 1040 stiffness (e.g., polyimide, stainless steel, NiTi) and/or coil pitch/diameter/material properties, braid parameters/material properties, etc., with or without having an additional proximal inner shaft body 1020 region.

In any of the embodiments described herein, the inner shaft 1010 contains an inner shaft stent region 1060 which is located at least partially under where the supplemental valve apparatus 10 is located with respect to the inner shaft 1010. The inner shaft stent region 1060 preferably has a lower profile than at least a portion of the inner shaft 1010 that is proximal to this region. It is desirable to have the inner shaft stent region 1060 with the loaded supplemental valve apparatus 10 and the outer shaft 1300 have sufficient flexibility to navigate the delivery system 1000 with supplemental valve apparatus 10 to the deployment location without abrupt changes in flexibility to improve tracking and avoid kinking. The inner shaft stent region 1060 can be constructed with one or more layers/components, including but not limited to an inner shaft stent region structural member 1080 and inner shaft stent region outer member 1090.

The inner shaft stent region structural member 1080 can provide resistance to compression while allowing the necessary flexibility to navigate the delivery system 1000 with supplemental valve apparatus 10 to the deployment location. The inner shaft stent region structural member 1080 can be comprised of, but not limited to, one or more tubular members, including a solid, fenestrated, or open tubular member, coil, and/or braid. The inner shaft stent region structural member 1080 may be a continuation of the proximal inner shaft body 1020. In these cases, the material, tube, braid, and/or coil properties may be changed to provide the desired functional characteristics. Examples include, for a braid, changing the braid pitch to increase flexibility, with a coil, reducing the wire diameter, changing material properties, changing the coil spacing, etc. may be used to increase flexibility, with a tube reducing the diameter and/or material properties, such as to be more flexible in the inner shaft stent region 1060.

The shaft stent region outer member 1090 may be made from a lubricious/low-friction material (e.g., high density polyethylene) to enable easy of release of the supplemental valve apparatus 10 during deployment.

The inner shaft stent region 1060 may be at least in part radiopaque and/or have one or more radiopaque markers 1150 to assist in identifying the location of the supplemental valve apparatus 10. There may be a proximal radiopaque marker 1150 located adjacent the proximal (with respect to the inner shaft 1010) end of the supplemental valve apparatus 10. In addition, there may be a distally located radiopaque marker 1150 adjacent the distal (with respect to the inner shaft 1010) end of the supplemental valve apparatus 10. An alternative or in addition to, the distal tip 1100 may be radiopaque and provide an indicator as to where the distal end of the supplemental valve apparatus 10 is positioned. The inner shaft stent region structural member 1080 may be radiopaque, such as made using a radiopaque material, for example, platinum, tantalum, iridium, gold, and their alloys or using a polymer (e.g., polyethylene, polyurethane, Pebax, nylon, blends) loaded with a radiopaque material (e.g., BaSO4). Inner shaft stent region outer member 1090 may be radiopaque, for example, using a polymer loaded with a radiopaque material as previously described for the inner shaft stent region structural member 1080.

In any of the embodiments described herein, the inner shaft 1010 and or outer shaft 1300 may include a liner 1012, such as PTFE, or any lubricious material or coating.

In any of the embodiments described herein, the inner shaft 1010 contains a distal tip 1100. It is desirable to have the distal tip 1100 contain the leading (most distal) end of the entire delivery system 1000. The distal tip 1100 is constructed to enable atraumatic and easy navigation of the anatomy, as well as provide a transition into more proximal regions of the inner shaft 1010 and/or outer shaft 1300.

In any of the embodiments described herein, the distal tip 1100 may include a distal tip taper 1110 and/or radiused distal tip distal end 1120. The distal tip 1100 may contain a region that is larger than outer profile or diameter as the distal end of the outer shaft 1300 to ensure the outer shaft 1300 does not have an exposed leading edge when introducing and navigating the delivery system 1000 into and through the anatomy.

In any of the embodiments described herein, the distal tip 1100 may have a bend or curve to the shape or be angled with respect to the longitudinal axis of more proximal regions of the inner shaft 1010. This is especially useful in constructions where there is no guide wire lumen, as the shape/angle will enable steering within the vasculature.

In any of the embodiments described herein, the distal tip 1100 contains a region with a distal tip taper 1110. This distal tip taper 1110 serves as a transition to the more proximal regions of the inner shaft 1000. The distal tip 1000 may be from approximately 0.5 cm up to 8 cm or more in length.

In any of the embodiments described herein, the distal tip 1000 contains a radius on the distal tip distal end 1120.

The distal tip 1100 can be made from one or more materials, for example, the distal tip 1100 may be constructed from a single polymer, e.g., polyethylene, polyurethane, blends, or be constructed with multiple polymers, such as blending or changing from one polymer to another to provide for a change in material stiffness and/or friction properties along the distal tip 1100.

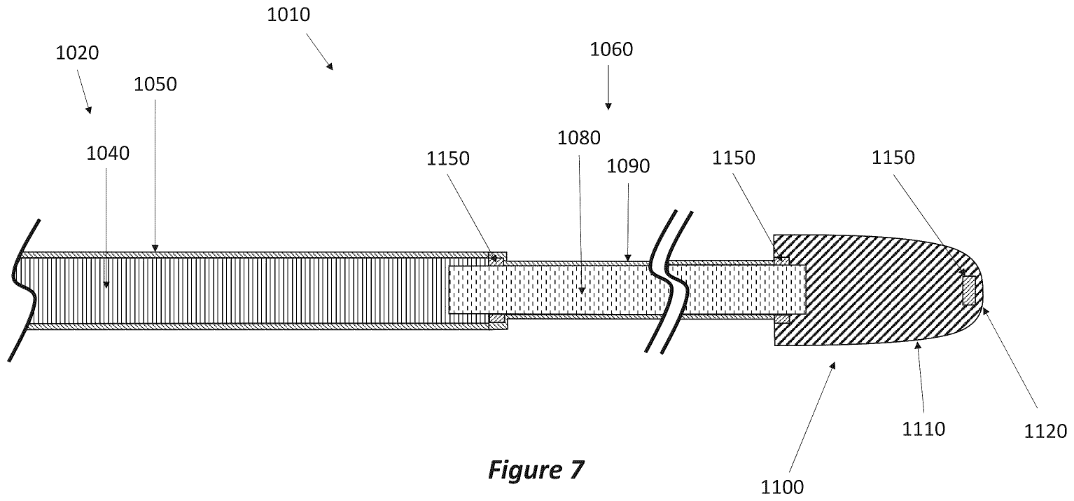

The inner shaft 1000 may be configured for introduction over a guide wire 3000 or may not include a guide wire lumen 1140 and be introduced without the use of a guide wire 3000. In embodiments using a guide wire 3000, the guide wire 3000 diameter may be from 0.010″ to 0.038″, more preferably from 0.025″ to 0.038″. An embodiment of an inner shaft 1000 without a guide wire lumen 1140 is depicted in FIG. 7.

In any of the embodiments described herein, the inner shaft 1010 surfaces may be coated or layered to increase or decrease friction/movement with respect to other delivery system 1000 surfaces and/or with respect to the guide wire 3000, vasculature, and/or other devices used during the procedure, such as guiding catheters, introducers, and the like. Surface coatings may be but not limited to hydrophilic, hydrophobic, fluoropolymers, silicone, polymer, etc.

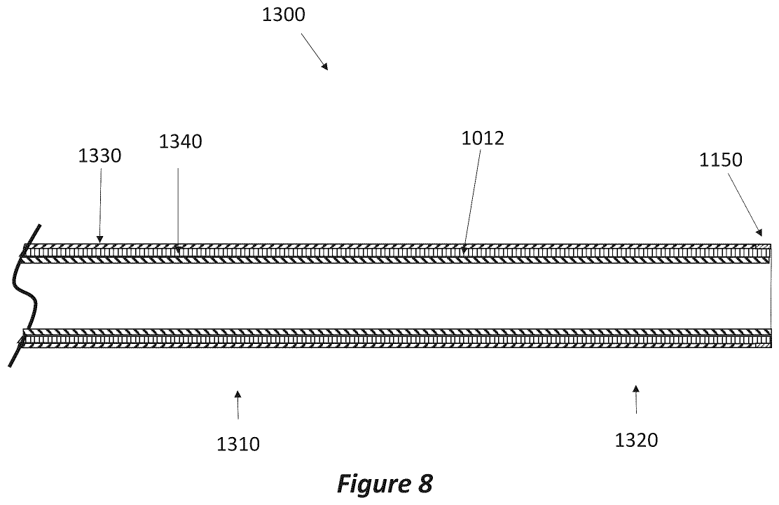

An embodiment of the outer shaft is depicted in FIG. 8. The outer shaft proximal region 1310 can be constructed with one or more sections to provide variable stiffness, resistance to elongation, and/or compressibility along the length. The outer shaft proximal region 1310 can be made from one or more layers/components, including but not limited to one or more of an outer shaft outer member 1330 and an outer shaft structural member 1340, or any individual component or combination thereof.

The outer shaft structural member 1340 generally resists elongation and can provide column strength to the outer shaft proximal region 1310. The outer shaft structural member 1340 can be made from, for example, one or more of the following: polymer, metal, polyimide, polyethylene, polyurethanes, polyamides and blends (e.g., Pebax®), stainless steel, NiTi, braided, coils, thermosets and thermoplastics.

The outer shaft stent region 1320 can be constructed with one or more sections to provide variable stiffness, resistance to elongation, and/or compressibility along the length. The outer shaft stent region 1320 can be made from one or more layers/components similar to that described for the outer shaft proximal region 1310.

The outer shaft 1300 inside surface is preferably lubricious/low-friction (e.g., high density polyethylene) with respect to the outer surface of the inner shaft 1010 over the range of motion of the outer shaft 1300 with respect to the inner shaft 1010. The outer shaft 1300 may include a liner 1012, typically made from a lubricious material (e.g., PTFE). This aides in the outer shaft 1300 being easily retracted with respect to the inner shaft 1010 during supplemental valve apparatus 10 deployment.

In any of the embodiments described herein, it may be desirable to change the flexibility of the outer shaft 1300 along the length. This may be accomplished by changing thickness or properties of materials, including the outer shaft structural member 1340 stiffness (e.g., polyimide, stainless steel, NiTi) and/or coil pitch/diameter/material properties, braid parameters/material properties, etc., with or without having an additional outer shaft proximal region 1310. The outer shaft proximal region 1310 may be constructed with two or more sections, such as the most proximal section that is comparatively less flexible, for example made with a NiTi hypotube outer shaft structural member 1340 and a more distal section that is comparatively more flexible, for example made with a coil for the outer shaft structural member 1340.

In any of the embodiments described herein, the outer shaft 1300 surfaces may be coated or layered to increase or decrease friction/movement with respect to other delivery system 1000 surfaces and/or with respect to the vasculature, and/or other devices used during the procedure, such as guiding catheters, introducers, and the like. Surface coatings may be but not limited to hydrophilic, hydrophobic, fluoropolymers, silicone, polymer, etc.

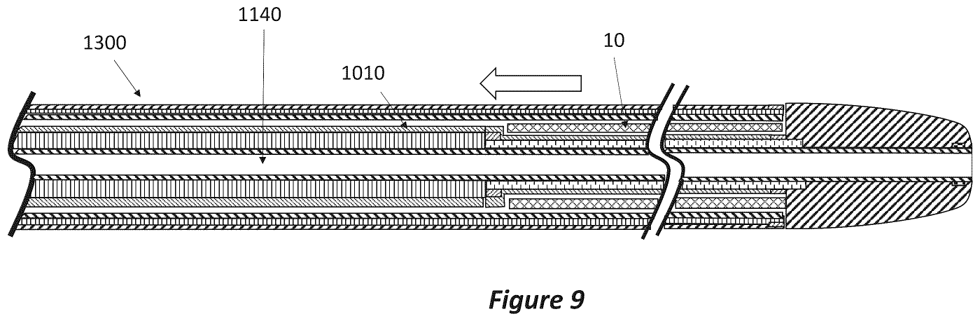

FIG. 9 depicts the supplemental valve apparatus 10 in position on a distal portion of the delivery system 1000. In this embodiment, the delivery system 1000 is configured where the outer shaft 1300 moves proximally with respect to the inner shaft 1010 to deploy the supplemental valve apparatus 10. Using this configuration, the supplemental valve apparatus 10 can be deployed from a superior approach, e.g., from the superior vena cava 50 via the jugular vein or by mounting the supplemental valve apparatus 10 on the delivery system 100 in the opposite longitudinal direction, from an inferior approach, e.g., from the inferior vena cava 60 via the femoral vein.

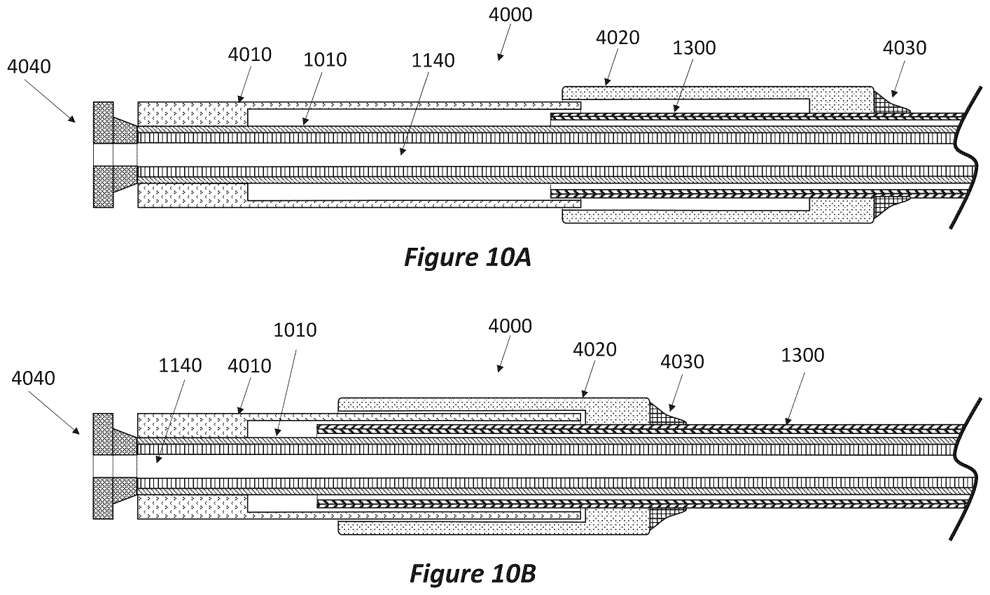

FIGS. 10A-B depict an embodiment of a handle 4000 with the delivery system 1000 of FIG. 9. The handle 4000 may have a base 4010 connected to the inner shaft 1010. The base 4010 may have a guide wire port 4040 in communication with a guide wire lumen 1140. The guide wire port 4040 may be or contain a Luer fitting. The handle 4000 also may have a slider 4020 connected to the outer shaft 1300 and may also have a strain relief 4030. FIG. 10A depicts the handle 4000 in a position with the supplemental valve apparatus 10 is constrained within the delivery system 1000. By retracting the slider 4020 with respect to the base 4010 (or vice versa), the outer shaft 1300 moves proximally with respect to the inner shaft 1010 deploying the supplemental valve apparatus 10 starting with the superior vena cava leg 300 and moving in a direction towards the inferior vena cava leg 500. FIG. 10B depicts the handle 4000 in a position with the slider 4020 and outer shaft 1300 fully retracted as it would be when the supplemental valve apparatus 10 has been fully released from the delivery system 1000.

An example configuration for a delivery system 1000 with an inner shaft 1010, and outer shaft 1300 and a handle 4000. The inner shaft 1010 includes a PTFE liner 1012 with a 0.038″ inside diameter and a 0.001″ wall thickness extending the length of the inner shaft 1010. The proximal inner shaft structural member 1040 consists of a stainless steel braid, with a round wire of 0.005″ extending the length of the inner shaft 1010, and a Pebax proximal inner shaft outer member 1050, laminated onto the proximal inner shaft structural member 1040 extending to the distal tip 1100. The distal tip 1100 is made from Pebax and bonded to the proximal inner shaft structural member 1040 and/or the proximal inner shaft outer member 1050. The distal tip 1100 is 5 cm in length, with a proximal outside diameter of 0.236″ tapering to an outer diameter of 0.100″ The proximal inner shaft outer member 1050 that is proximal to the supplemental valve apparatus 10 may have an increased diameter. The outside diameter of the proximal inner shaft outer member 1050 that is under the supplemental valve apparatus 10 has an outside diameter of 0.080″.

The outer shaft 1300 includes a PTFE liner 1012 with a 0.200″ inside diameter and a 0.001″ wall thickness extending the length of the outer shaft 1300. The outer shaft structural member 1340 consists of a stainless steel braid, with a round wire of 0.004″ extending the length of the outer shaft 1300, and a Pebax proximal outer shaft outer member 1330 laminated onto the outer shaft structural member 1340, Radiopaque markers 1150 are positioned on the inner shaft 1010 adjacent the ends of the supplemental valve apparatus 10, adjacent the distal end of the distal tip 1100, and on the outer shaft 1300 adjacent the distal end of the outer shaft 1300.

The handle 4000 is molded and enables the outer shaft 1300 to be retracted with respect to the inner shaft 1010.

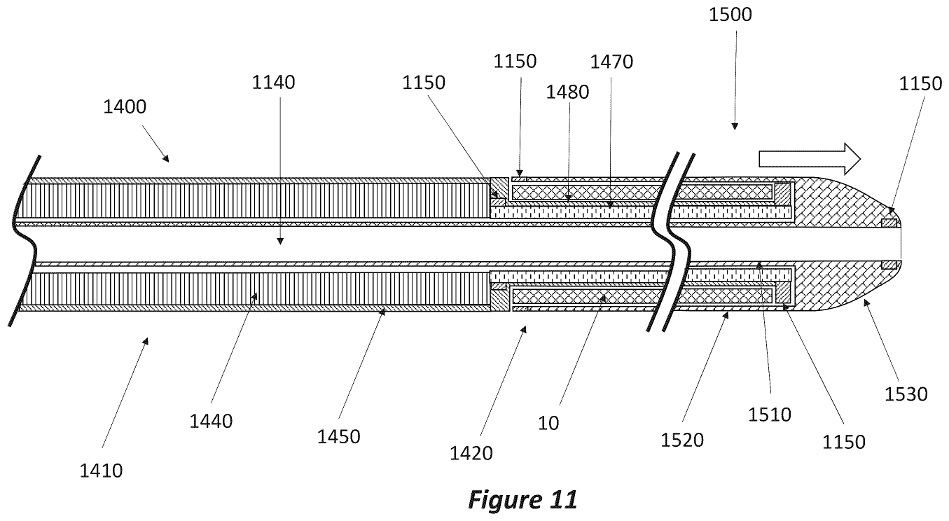

FIG. 11 depicts an embodiment of the device wherein the proximally mounted region of the supplemental valve apparatus 10 is deployed first with respect to the delivery system 1000. In this embodiment, there is a proximal shaft 1400 and a distal shaft 1500. The distal shaft 1500 moves distally relative to the proximal shaft 1400, thus enabling the proximally mounted portion of the supplemental valve apparatus 10 to be uncovered and deployed first and then continuing to deploy towards the distal end. This embodiment is typically suitable for an inferior approach where it is desired to deploy the inferior vena cava leg 500 first, enabling accurate positioning of the inferior vena cava leg 500 with respect to the hepatic vein(s) 70.

The proximal shaft 1400 can be constructed with one or more sections to provide variable stiffness, resistance to elongation, and/or compressibility along the length, such as a proximal shaft proximal region 1410 and a proximal shaft stent region 1420. The proximal shaft proximal region 1410 can be made from a combination of a proximal shaft outer member 1450 and a proximal shaft structural member 1440 or any additional layers and/or individual component or combination thereof, such as the proximal shaft proximal region 1410 being made from a PTFE impregnated polyimide with or without a proximal shaft outer member 1450.

The proximal shaft structural member 1440 generally resists elongation and can provide column strength to the proximal shaft proximal region 1410. The proximal shaft structural member 1440 can be made from, for example, one or more of the following: polymer, metal, polyimide, polyethylene, polyurethanes, polyamides and blends (e.g., Pebax®), stainless steel, NiTi, braided, coils, thermosets and thermoplastics.

The proximal shaft proximal region 1410 inside surface is preferably lubricious/low-friction (e.g., high density polyethylene) with respect to the outer surface of the distal shaft inner member 1510 over the range of motion of the proximal shaft 1400 with respect to the distal shaft inner member 1510. This aides in the distal shaft 1500 being easily advanced with respect to the proximal shaft 1400 during supplemental valve apparatus 10 deployment.

In any of the embodiments described herein, it may be desirable to change the flexibility of the proximal shaft 1400 along the length. This may be accomplished by changing thickness or properties of materials, including the proximal shaft structural member 1440 stiffness (e.g., polyimide, stainless steel, NiTi) and/or coil pitch/diameter/material properties, braid parameters/material properties, etc., with or without having an additional proximal shaft proximal region 1410. The proximal shaft proximal region 1410 may be constructed with two or more sections, such as the most proximal section that is comparatively less flexible, for example made with a NiTi hypotube for the proximal shaft structural member 1440 and a more distal section that is comparatively more flexible, for example made with a coil for the proximal shaft structural member 1440.

In any of the embodiments described herein, the proximal shaft 1400 surfaces may be coated or layered to increase or decrease friction/movement with respect to other delivery system 1000 surfaces and/or with respect to the vasculature, and/or other devices used during the procedure, such as guiding catheters, introducers, and the like. Surface coatings may be but not limited to hydrophilic, hydrophobic, fluoropolymers, silicone, polymer, etc.

The proximal shaft stent region 1420 may be constructed similar to the proximal shaft proximal region 1410, for example with a proximal shaft stent region outer member 1480 and a proximal shaft stent region structural member 1470 or any individual component or combination thereof. The proximal shaft stent region 1420 typically has a smaller outside diameter than the proximal shaft proximal region 1410, to accommodate placement of the supplemental valve apparatus 10.

The distal shaft 1500 can be constructed of a distal shaft inner member 1510, distal shaft outer member 1520, distal shaft tip 1530, including where one or more of these can be combined into a single component. The distal shaft inner member 1510 is functionally connected to the distal shaft outer member 1520, which may be accomplished by being in directly coupled to each other or by having one or more intermediate components, for example, having the distal shaft inner member 1510 and the distal shaft outer member both coupled to the distal shaft tip 1530.

The distal shaft inner member 1510 preferably has relatively high resistance to compression, such that the distal shaft outer member 1520 moves relative to the distal shaft inner member 1510 when deploying the supplemental valve apparatus 10. The distal shaft inner member 1510 may be constructed from a single material or combination of materials and components, for example, one or more of polyimide, FTFE impregnated polyimide, polyethylene, polyurethane, co-polymers, braid, coil, hypotube, etc.

The distal shaft outer member 1520 in this embodiment covers the supplemental valve apparatus 10 and when moved distally with respect to the supplemental valve apparatus 10 to deploy the supplemental valve apparatus 10 from the proximal to the distal end. The distal shaft outer member 1520 may be constructed from a single material or combination of materials and components, for example, one or more of polyimide, FTFE impregnated polyimide, polyethylene, polyurethane, co-polymers, braid, coil, hypotube, etc. The distal shaft outer member 1520 may also have a liner 1012, similar to what was described for the proximal shaft 1400.

The distal shaft tip 1530 preferably includes the distal aspect of the delivery system 1000. The distal shaft tip 1530 is atraumatic, generally but not limited to being smaller in diameter at the distal end than at the proximal end with radiuses at any changes in profile so as not to contain sharp edges which may cause tissue damage.

Radiopaque markers 1150 and/or radiopaque components/regions are preferably used to identify the distal end of the distal shaft tip 1530 as well as the location of the supplemental valve apparatus 10. Radiopaque markers 1150 may be made from, for example, platinum, tantalum, iridium, gold, and their alloys. In place of or in addition to, the distal shaft tip 1530 and/or the distal shaft outer member 1520 and/or the proximal shaft stent region structural member 1470 and/or the proximal shaft stent region outer member 1480 may be made from or contain radiopaque materials, such as using a polymer (e.g., polyethylene, polyurethane, Pebax, nylon, blends) loaded with a radiopaque material (e.g., BaSO₄).

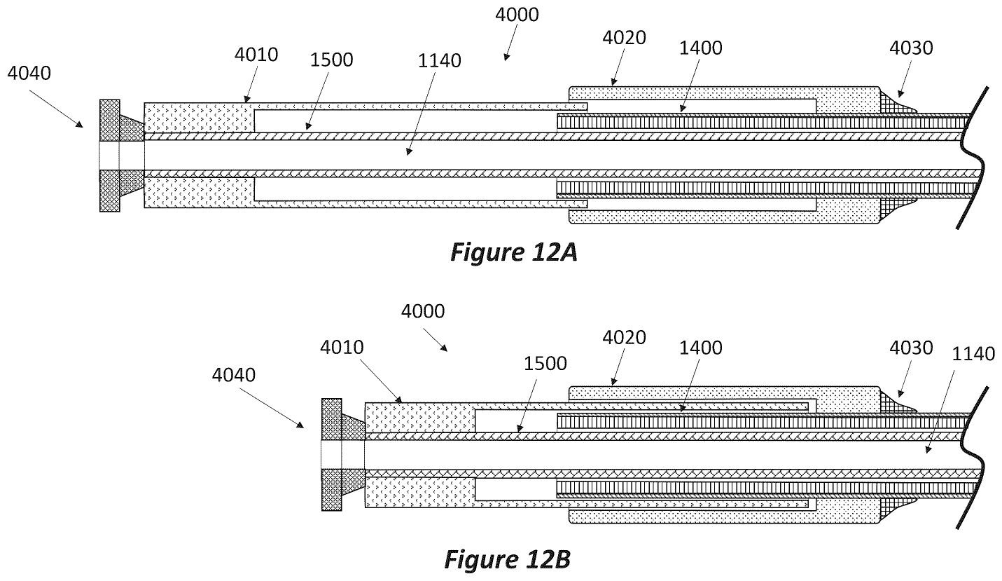

FIGS. 12A-B depict an embodiment of a handle 4000 with the delivery system 1000 of FIG. 11. The handle 4000 may have a base 4010 connected to the distal shaft 1500. The base 4010 may have a guide wire port 4040 in communication with a guide wire lumen 1140. The guide wire port 4040 may be or contain a Luer fitting. The handle 4000 also may have a slider 4020 connected to the proximal shaft 1400 and may also have a strain relief 4030. FIG. 12A depicts the handle 4000 in a position with the supplemental valve apparatus 10 is constrained within the delivery system 1000. By advancing the base 4010 with respect to the slider 4020 (or vice versa), the distal shaft 1500 moves distally with respect to the proximal shaft 1400 deploying the supplemental valve apparatus starting with the inferior vena cava leg 500 and moving in a direction towards the superior vena cava leg 300. FIG. 12B depicts the handle 4000 in a position with the base 4010 and distal shaft 1500 as it would be when the supplemental valve apparatus 10 has been fully released from the delivery system 1000.

An example configuration for a delivery system 1000, includes a distal shaft 1500, a proximal shaft 1400, and a handle 4000. The distal shaft 1500 includes a distal shaft inner member 1510, distal shaft outer member 1520, and distal shaft tip 1530. The distal shaft inner member 1510 includes a PTFE liner 1012 with a 0.038″ inside diameter and a 0.001″ wall thickness extending the length of the distal shaft 1500. The distal shaft inner member 1510 includes a stainless steel braid, with a round wire of 0.005″ extending the length of the distal shaft inner member 1510, and a Pebax jacket laminated over the braid, extending to the distal tip 1100. The distal shaft inner member 1510 has an inside diameter of 0.038″ and an outside diameter of 0.085″. The distal tip 1100 is 5 cm in length, with a proximal outside diameter of 0.260″ tapering to an outer diameter of 0.100″. The distal shaft outer member 1520 extends proximally from the distal tip 1100 to cover the supplemental valve apparatus 10. The distal shaft outer member 1520 has a PTFE liner 1012 with an inside diameter of 0.220″ and a wall thickness of 0.001″. The outside diameter of the distal shaft outer member 1520 is 0.260″.

The proximal shaft 1400 includes a proximal shaft structural member 1440, a proximal shaft outer member 1450, proximal shaft stent region structural member 1470, and a proximal shaft stent region outer member 1480. The proximal shaft structural member 1440 is made from a NiTi tube, the inside diameter of the proximal shaft structural member 1440 is 0.090″ with an outside diameter of 0.180″. The proximal shaft outer member 1450 is made of Pebax, is hydrophilically coated, and is coupled to the proximal shaft structural member 1440. The proximal shaft structural member 1440 has an outside diameter of 0.260″. The proximal shaft stent region structural member 1470 made from Pebax with an inside diameter of 0.090″ and an outside diameter of 0.110″. A proximal shaft stent region outer member 1480 is coupled to the proximal shaft stent region structural member 1470 and has an outside diameter of 0.120″.

The proximal shaft 1400 includes radiopaque markers 1150 positioned on the outer shaft 1400 adjacent where the ends of the supplemental valve apparatus 10 will lie, with the distal radiopaque marker 1150 forming a stop to prevent the supplemental valve apparatus 10 from moving distally during deployment, as well as a radiopaque markers 1150, adjacent the distal end of the distal tip 1100, and on the outer shaft 1300 adjacent the proximal end of the distal shaft 1500.

The handle 4000 is molded and enables the distal shaft 1500 to be advanced with respect to the proximal shaft 1400.

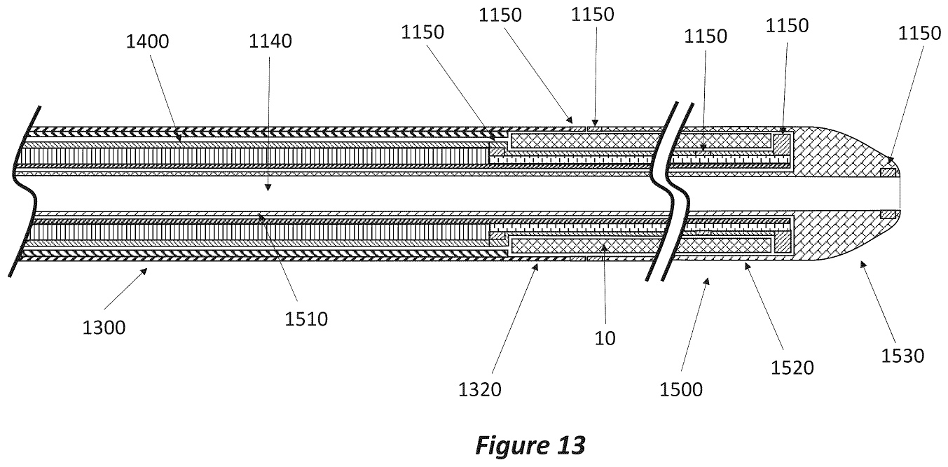

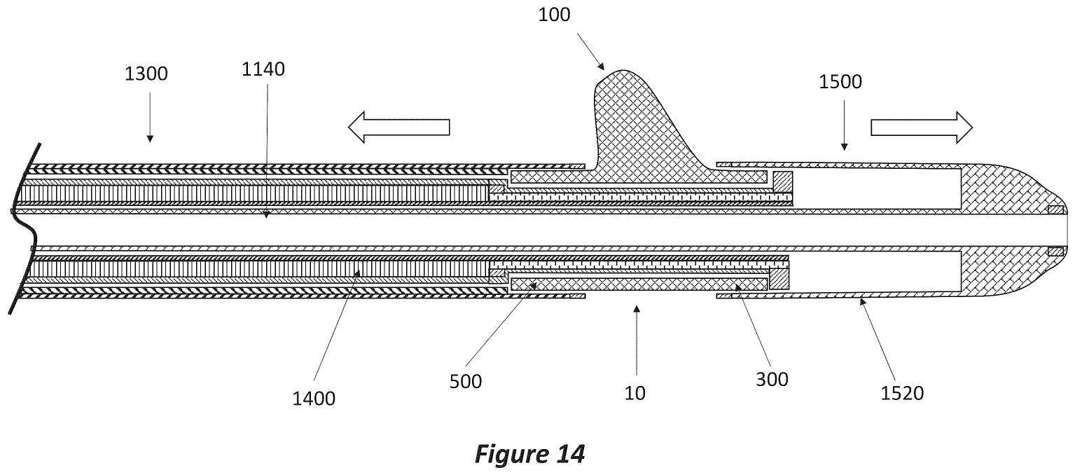

FIGS. 13 and 14 depict an embodiment where the supplemental valve leg 100 can be deployed first, followed by either the superior vena cava leg 300 or inferior vena cava leg 500. The advantage of deploying the supplemental valve leg 100 first allows for the rotational and/or longitudinal orientation to be optimized, then either the superior vena cava leg 300 or inferior vena cava leg 500 to be adjusted to the optimum position in the corresponding vessel and then deployed. Following that, the remaining leg can be deployed.

As depicted, the distal shaft 1500 has a distal shaft outer member 1520 that extends over the length of the supplemental valve leg 100. The distal shaft 1500 moves distally relative to the proximal shaft 1400, thus enabling the supplemental valve leg 100 to be uncovered and deployed first, as depicted in FIG. 14. In another configuration, the proximal shaft 1400 may be moved proximally to deploy the supplemental valve leg 100. This depends on if the delivery system 1000 is configured with the supplemental valve leg 100 captured by the distal shaft 1500, proximal shaft 1400, or both. After the supplemental valve leg 100 is deployed, the rotational orientation of the supplemental valve leg 100 and supplemental valve 130 can be optimized. In addition, longitudinal orientation with respect to the superior vena cava leg 300 and/or inferior vena cava leg 500 can be adjusted. Either the superior vena cava leg 300 or inferior vena cava leg 500 is then deployed. The supplemental valve 130 can then be tested as well as the seal on the deployed superior vena cava leg 300 or inferior vena cava leg 500. If adjustment needs to be done, the deployed leg and if desired, the supplemental valve leg 100 can be recaptured by the delivery system 1000. Once satisfactory placement of the supplemental valve leg 100 and the initially deployed leg is achieved, the remaining leg may be deployed. At this point, the entire supplemental valve apparatus 10 can be tested.

In this configuration, the distal shaft outer member 1520 does not extend over the entire length of the supplemental valve apparatus 10, but only a portion of it. The outer shaft 1300 contains an outer shaft stent region 1320 which covers the remaining portion of the supplemental valve apparatus 10, to fully capture the supplemental valve apparatus 10.

Construction of the proximal shaft 1400 can be as previously described, with the outside diameter properly sized to fit inside the outer shaft 1300 and the inside diameter sized to fit over the distal shaft inner member 1510.

Construction of the outer shaft 1300 can be as preciously described with an outer shaft stent region 1320 that does not extend over the entire length of the supplemental valve apparatus 10 and adjusting diameters as appropriate.

Construction of the distal shaft 1500 can be as previously described with a distal shaft outer member 1520 that does not extend over the entire length of the supplemental valve apparatus 10.

Radiopaque markers 1520 may be positioned to identify the ends of the outer shaft 1300 and the distal shaft outer member 1520, as well as the distal shaft tip 1530, and adjacent the ends of the supplemental valve apparatus 10 which may be located on any of the shafts in the area.

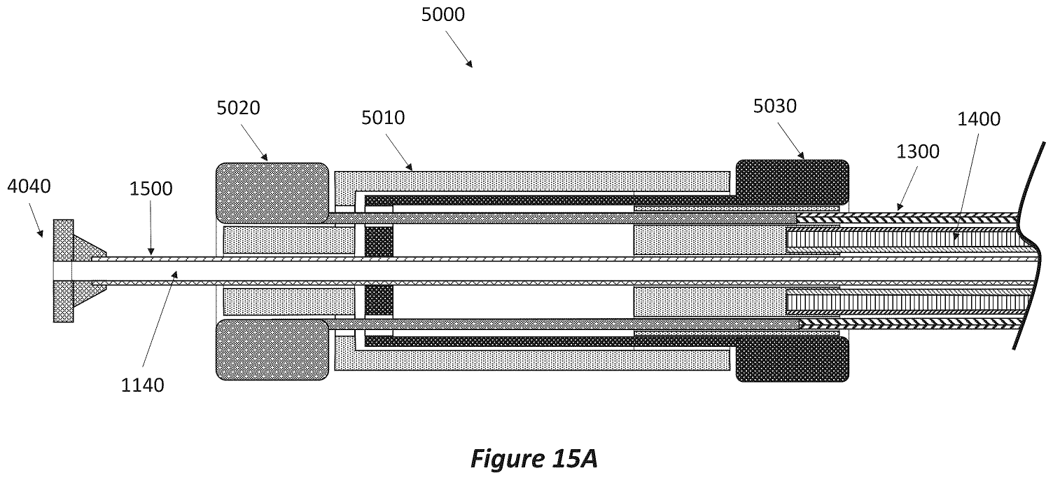

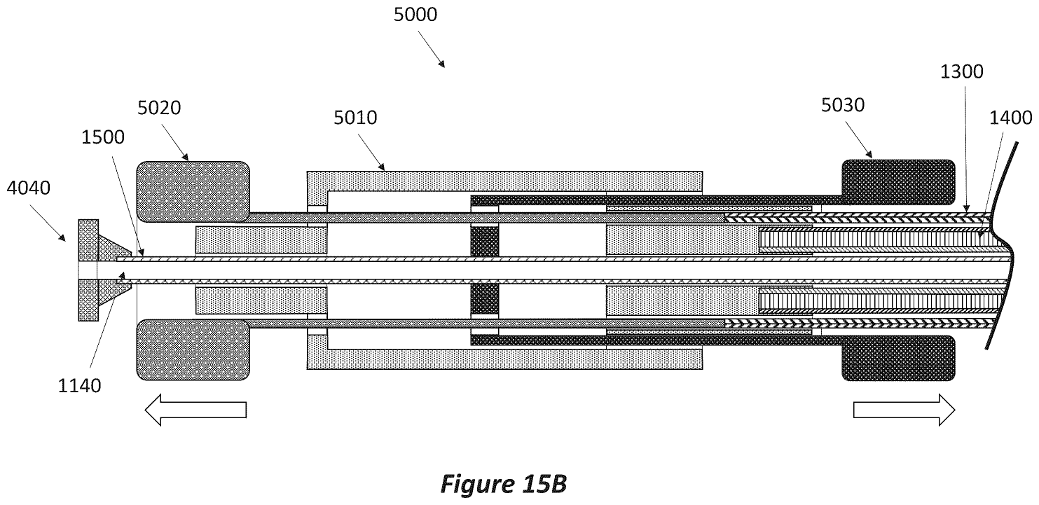

FIGS. 15A-B depict an embodiment of a dual-action handle 5000 with the delivery system 1000 of FIG. 13 and FIG. 14. The dual-action handle 5000 may have a mid-body 5010 connected to the proximal shaft 1400, a proximal deployment slider 5020 connected to the outer shaft 1300, and a distal deployment slider 5030 connected to the distal shaft 1500. The proximal deployment slider 5020 and distal deployment slider 5030 may be connected to their respective shafts having a configuration that allows the outer shaft 1300 to be connected to the proximal deployment slider 5020 with the gripping portion of the proximal deployment slider 5020 located proximally of the mid-body 5010, as well as the distal deployment slider 5030 connected to the distal shaft 1500 with the gripping portion of the distal deployment slider 5030 located distally of the mid-body 5010. This configuration allows the supplemental valve leg 100 to be deployed first, either by moving the distal deployment slider 5030 distally and/or the proximal deployment slider 5020 proximally. This is followed, in either order, by moving the distal deployment slider 5030 distally to deploy the superior vena cava leg 300 and the proximal deployment slider 5020 proximally to deploy the inferior vena cava leg 500.

An example configuration for a delivery system 1000, includes a distal shaft 1500, a proximal shaft 1400, outer shaft 1300, and a handle 5000. The distal shaft 1500 includes a distal shaft inner member 1510, distal shaft outer member 1520, and distal shaft tip 1530. The distal shaft inner member 1510 includes a PTFE liner with a 0.038″ inside diameter and a 0.001″ wall thickness extending the length of the distal shaft 1500. The distal shaft inner member 1510 includes a stainless steel braid, with a round wire of 0.005″ extending the length of the distal shaft inner member 1510, and a Pebax jacket laminated over the braid, extending to the distal tip 1100. The distal shaft inner member 1510 has an inside diameter of 0.038″ and an outside diameter of 0.068″. The distal tip 1100 is 5 cm in length, with a proximal outside diameter of 0.286″, tapering to an outer diameter of 0.100″. The distal shaft outer member 1520 extends proximally from the distal tip 1100 to cover a portion of the supplemental valve apparatus 10. The distal shaft outer member 1520 has a PTFE liner 1012 with an inside diameter of 0.246″ and a wall thickness of 0.001″. The outside diameter of the distal shaft outer member 1520 is 0.286″.

The proximal shaft 1400 includes a proximal shaft structural member 1440, a proximal shaft outer member 1450, proximal shaft stent region structural member 1470, and a proximal shaft stent region outer member 1480. The proximal shaft structural member 1440 is made from a NiTi tube, the inside diameter of the proximal shaft structural member 1440 is 0.075″ with an outside diameter of 0.140″. The proximal shaft outer member 1450 is made of Pebax, is hydrophilically coated, and is coupled to the proximal shaft structural member 1440. The proximal shaft structural member 1440 has an outside diameter of 0.180″. The proximal shaft stent region structural member 1470 made from Pebax with an inside diameter of 0.075″ and an outside diameter of 0.090″. A proximal shaft stent region outer member 1480 is coupled to the proximal shaft stent region structural member 1470 and has an outside diameter of 0.100″.

The proximal shaft 1400 includes radiopaque markers 1150 positioned on the outer shaft 1400 adjacent where the ends of the supplemental valve apparatus 10 will lie, with the distal radiopaque marker 1150 forming a stop to prevent the supplemental valve apparatus 10 from moving distally during deployment, as well as a radiopaque markers, adjacent the distal end of the distal tip 1100, and on the outer shaft 1300 adjacent the distal end of the outer shaft 1300.

The outer shaft 1300 includes a PTFE liner 1012 with a 0.230″ inside diameter and a 0.001″ wall thickness extending the length of the outer shaft 1300. The outer shaft structural member 1340 consists of a stainless steel braid, with a round wire of 0.004″ extending the length of the outer shaft 1300, and a Pebax proximal outer shaft outer member 1330 laminated onto the outer shaft structural member 1340 with an outside diameter of 0.286″, Radiopaque markers 1150 are positioned on the distal shaft 1500 adjacent the distal end of the distal tip 1100, and on the outer shaft 1300 adjacent the distal end of the outer shaft 1300, on the proximal shaft 1400 adjacent the ends of the location of the supplemental valve apparatus 10, and on the outer shaft 1300 adjacent the distal end of the outer shaft 1300.

The handle 5000 is molded and enables the distal shaft 1500 to be advanced with respect to the proximal shaft 1400 and the outer shaft 1300 to be retracted with respect to the proximal shaft 1400.

Handles (4000, 5000) may be fabricated using any techniques, such as molding, machining, etc. and from suitable materials, including polymers, metals, composites, which may include polycarbonate, polyurethane, amides, etc. Handles (4000, 5000) for the delivery system 1000 do not need to be based on a slide mechanism. Any appropriate designs may be used to deploy the supplemental valve apparatus 10 in any of the manners previously described. Example mechanisms include gear drive, pulley, screw, pin and pull, as long as the shafts can be moved with respect to each other.

The above embodiments were depicted for an inferior vena cava 60 approach (femoral). In any of the delivery systems 1000, the supplemental valve apparatus 10 may be mounted/constrained within the delivery system 1000 in either direction. This enables an approach from above the superior vena cava 50 (jugular approach) to be able to deploy the superior vena cava leg 300 first when using a delivery system 1000 as depicted in FIG. 11 and the supplemental valve apparatus 10 mounted with the superior vena cava leg 300 towards the proximal end of the delivery system 1000. Similarly, from above the superior vena cava 50 (jugular approach) the inferior vena cava leg 500 can be deployed first when using a delivery system 1000 as depicted in FIG. 11 and the supplemental valve apparatus 10 mounted with the inferior vena cava leg 500 towards the distal end of the delivery system 1000. Using a delivery system 1000 similar to that depicted in FIG. 13 and FIG. 14, the mounting direction of the supplemental valve apparatus 10 provides for either an superior vena cava 50 approach or inferior vena cava 60 (femoral) approach.

The supplemental valve apparatus 10 and delivery system 1000 and/or individual components or any subset of the components described herein can be used as a complete system, individually, in combinations, and/or with other guidewires, catheters, and vascular and non-vascular devices. Various sizes and combinations can be selected and used depending upon the intended clinical procedure.

### Examples

The present technology is illustrated, for example, according to various aspects described below. Various examples of aspects of the present technology are described as numbered examples (1, 2, 3, etc.) for convenience. These are provided as examples and do not limit the present technology. It is noted that any of the dependent examples may be combined in any combination and placed into a respective independent example. The other examples can be presented in an equivalent manner.
1. A supplemental valve apparatus for implantation into the right atrium, comprising: a supplemental valve; and the supplemental valve coupled to at least one supplemental valve structure, the supplemental valve structure extends into at least one of the inferior vena cava or superior vena cava; and the supplemental valve being positioned in or adjacent the right atrium.
2. The supplemental valve apparatus of example 1, and wherein: the supplemental valve is a biologic valve.
3. The supplemental valve apparatus of example 1, and wherein: the supplemental valve apparatus is positioned within the right atrium, and anchored in position by a structure extending into the inferior vena cava and into the superior vena cava.
4. The supplemental valve apparatus of example 1, and wherein: the supplemental valve apparatus includes one or more of a jacket and/or seal(s) to direct blood flow from the inferior vena cava and superior vena cava through the supplemental valve.
5. The supplemental valve apparatus of example 1, and wherein: the supplemental valve apparatus is configured to enable percutaneous delivery via a delivery system.
6. The supplemental valve apparatus of example 1, and wherein: the supplemental valve apparatus includes a delivery system for deploying a supplemental valve into the right atrium, comprising: an inner shaft; an outer shaft; a supplemental valve apparatus positioned between the inner shaft and the outer shaft, the outer shaft configured to be retracted with respect to the inner shaft to deploy the supplemental valve apparatus; the supplemental valve apparatus having a supplemental valve, an inferior vena cava leg for anchoring at least a portion of the supplemental valve apparatus in the inferior vena cava, and a superior vena cava leg, for anchoring at least a portion of the supplemental valve apparatus in the superior vena cava.
7. The supplemental valve apparatus of example 1, and wherein: the supplemental valve apparatus includes a delivery system for deploying a supplemental valve into the right atrium, comprising: a distal shaft; a proximal shaft; a supplemental valve apparatus positioned between the distal shaft and the proximal shaft, the distal shaft configured to be advanced with respect to the proximal shaft to deploy the supplemental valve apparatus; the supplemental valve apparatus having a supplemental valve, an inferior vena cava leg for anchoring at least a portion of the supplemental valve apparatus in the inferior vena cava, and a superior vena cava leg, for anchoring at least a portion of the supplemental valve apparatus in the superior vena cava.
8. The supplemental valve apparatus of example 1, and wherein: the supplemental valve apparatus includes a delivery system for deploying a supplemental valve into the right atrium, comprising: a distal shaft; a proximal shaft; an outer shaft; a supplemental valve apparatus positioned between at least a portion of the distal shaft inner member and the outer shaft, the outer shaft configured to be retracted with respect to the distal shaft inner member to deploy at least a portion of the supplemental valve apparatus; the distal shaft configured to be advanced with respect to the distal shaft inner member to deploy at least a portion of the supplemental valve apparatus; the supplemental valve apparatus having a supplemental valve, and at least one of an inferior vena cava leg for anchoring at least a portion of the supplemental valve apparatus in the inferior vena cava, and a superior vena cava leg, for anchoring at least a portion of the supplemental valve apparatus in the superior vena cava.
9. A method for treating a compromised tricuspid valve, comprising: a supplemental valve apparatus including a supplemental valve; leaving the existing tricuspid valve in position; placing a supplemental valve in or adjacent the right atrium; maintaining the position of the supplemental valve with a structure located at least in part within the superior vena cava and/or the inferior vena cava; and directing blood flow from the superior vena cava and inferior vena cava through the supplemental valve.
10. The method of example 9 wherein the supplemental valve apparatus is delivered to the desired location percutaneously.
11. The method of example 9, and wherein: the supplemental valve apparatus is deployed at least in part by retracting an outer shaft.
12. The method of example 9, and wherein: the supplemental valve apparatus is deployed at least in part by advancing a distal shaft.
13. The method of example 9, and wherein: the supplemental valve apparatus is deployed at least in part by advancing a distal shaft and retracting an outer shaft.
14. The method of example 9, and wherein: the supplemental valve is deployed in or adjacent the right atrium, and then the supplemental valve apparatus is anchored in position.

### 15. Example Procedure—Superior Vena Cava Leg Deployed First

The following example describes one procedure for deploying the supplemental valve apparatus 10 in the right atrium 20 (and superior vena cava 50 and inferior vena cava 60). The procedure describes a femoral (below the inferior vena cava 60) approach and places the superior vena cava leg 300 first, followed by the supplemental valve leg 100 and then the inferior vena cava leg 500. The superior vena cava leg 300 is positioned in the delivery system 1000 in the distal location on a delivery system 1000 depicted in FIG. 9 with a handle 4000 as depicted in FIGS. 10A-B.

Femoral access is obtained following normal percutaneous or cutdown procedures. A guide wire 3000 is introduced and advanced into the superior vena cava 50. If using a delivery system 1000 similar to that depicted in FIG. 7, no guide wire 3000 is used. The delivery system 1000 is advanced over the guide wire 3000 until the radiopaque element 410 for the superior vena cava leg 300 is in the desired location. The delivery system 1000 is rotated until the radiopaque element 410 for the supplemental valve leg 100 is oriented toward the right atrium 20 and native tricuspid valve 30. The slider 4020 is retracted enabling the superior vena cava leg 300 of the supplemental valve apparatus 100 to begin deployment and engage the superior vena cava 50. The supplemental valve leg 100 may also be deployed into the right atrium 20. The supplemental valve 130 and superior vena cava leg sealing cuff 320 may be tested for functionality and sealing. If the positioning or seal is undesirable, the slider 4020 may be advanced to recapture the supplemental valve apparatus 100 with subsequent repositioning and retraction of the slider 4020 until satisfactory positioning has been achieved. Continuing to retract the slider 4020, deploys the inferior vena cava leg sealing cuff 520, and then the rest of the inferior vena cava leg 500. The supplemental valve apparatus 100 is now in place. Normal visualization methods may be used to ensure the placement and seals are correct, as well as the function of the supplemental valve 130. The delivery system 1000 is retracted from the patient, the guide wire 3000 removed, and the access site closed following normal procedures.

### 16. Example Procedure—Inferior Vena Cava Leg Deployed First

The following example describes one procedure for deploying the supplemental valve apparatus 10 in the right atrium 20 (and superior vena cava 50 and inferior vena cava 60). The procedure describes a femoral (below the inferior vena cava 60) approach and places the inferior vena cava leg 500 first, followed by the supplemental valve leg 100 and then the superior vena cava leg 300. The superior vena cava leg 300 is positioned in the delivery system 1000 in the distal location on a delivery system 1000 depicted in FIG. 11 with a handle 4000 as depicted in FIGS. 12A-B.

Femoral access is obtained following normal percutaneous or cutdown procedures. A guide wire 3000 is introduced and advanced into the superior vena cava 50. If using a distal shaft 1500 similar to that depicted in FIG. 7, no guide wire 3000 is used. The delivery system 1000 is advanced over the guide wire 3000 until the radiopaque element 410 for the inferior vena cava leg 500 is in the desired location, allowing for the inferior vena cava leg sealing cuff 520 to be placed above the hepatic vein 70 (see FIG. 1). The delivery system 1000 is rotated until the radiopaque element 410 for the supplemental valve leg 100 is oriented toward the right atrium 20 and native tricuspid valve 30. The base 4010 is advanced with respect to the slider 4020 enabling the inferior vena cava leg 500 of the supplemental valve apparatus 100 to begin deployment and engage the inferior vena cava 60. The supplemental valve leg 100 may also be deployed into the right atrium 20. The supplemental valve 130 and inferior vena cava leg sealing cuff 520 may be tested for functionality and sealing. If the positioning is undesirable, the base 4010 may be retracted to recapture the supplemental valve apparatus 100 with subsequent repositioning and advancement of the base 4010 until satisfactory positioning has been achieved. Continuing to advance the base 4010, deploys the superior vena cava leg sealing cuff 320, and then the rest of the superior vena cava leg 300. As the supplemental valve apparatus 100 is fully deployed, the anchors 400 on the superior vena cava leg 300 engage the superior vena cava 50. The supplemental valve apparatus 100 is now in place. Normal visualization methods may be used to ensure the placement and seals are correct, as well as the function of the supplemental valve 130. The delivery system 1000 is retracted from the patient, the guide wire 3000 removed, and the access site closed following normal procedures.

### 17. Example Procedure—Supplemental Valve Leg Deployed First

The following example describes one procedure for deploying the supplemental valve apparatus 10 in the right atrium 20 (and superior vena cava 50 and inferior vena cava 60). The procedure describes a femoral (below the inferior vena cava 60) approach and places the supplemental valve leg 100 first, followed by the superior vena cava leg 300 and then the inferior vena cava leg 500. Either the superior vena cava leg 300 or inferior vena cava leg 500 may be placed first, depending on the preference of the physician. The superior vena cava leg 300 is positioned in the delivery system 1000 in the distal location on a delivery system 1000 depicted in FIG. 13 and FIG. 14 with a dual-action handle 5000 as depicted in FIGS. 15A-B.

Femoral access is obtained following normal percutaneous or cutdown procedures. A guide wire 3000 is introduced and advanced into the superior vena cava 50. If using a distal shaft 1500 similar to that depicted in FIG. 7, no guide wire 3000 is used. The delivery system 1000 is advanced over the guide wire 3000 until the radiopaque element 410 for the superior vena cava leg 300 is in the desired location. The delivery system 1000 is rotated until the radiopaque element 410 for the supplemental valve leg 100 is oriented toward the right atrium 20 and native tricuspid valve 30. While holding the mid-body 5010 in position, the distal deployment slider 5030 is advanced with respect to the mid-body 5010 enabling the supplemental valve leg 100 to begin deployment. Orientation may now be obtained by rotating the delivery system 1000 and using the supplemental valve leg 100 radiopaque element 410. In addition, the superior vena cava leg 300 may be placed in the desired location. The distal deployment slider 5030 is further advanced, enabling the superior vena cava leg 300 of the supplemental valve apparatus 100 to begin deployment and engage the superior vena cava 50. If the positioning is undesirable, the distal deployment slider 5030 may be retracted to recapture the supplemental valve apparatus 100 with subsequent repositioning and advancement of the distal deployment slider 5030 until satisfactory positioning has been achieved. Continuing to advance the distal deployment slider 5030, deploys the superior vena cava leg sealing cuff 320 and then fully deploys the rest of the superior vena cava leg 300. The supplemental valve 130 and superior vena cava leg sealing cuff 320 may be testing for functionality and sealing. If the positioning or seal is undesirable, the distal deployment slider 5030 may be retracted to recapture the supplemental valve apparatus 100 with subsequent repositioning and advancement of the distal deployment slider 5030.

While holding the mid-body 5010 in position, the proximal deployment slider 5020 is retracted, deploying the inferior vena cava leg sealing cuff 520, and then the inferior vena cava leg 500. As the supplemental valve apparatus 100 is fully deployed, the inferior vena cava leg 500 engage the inferior vena cava 60. The supplemental valve apparatus 100 is now in place. Normal visualization methods may be used to ensure the placement and seals are correct, as well as the function of the supplemental valve 130. The delivery system 1000 is retracted from the patient, the guide wire 3000 removed, and the access site closed following normal procedures.

Alternate embodiments may be devised without departing from the spirit or the scope of the present technology. Additionally, well-known elements of embodiments of the systems, apparatuses, and methods have not been described in detail or have been omitted so as not to obscure the relevant details of the systems, apparatuses, and methods.

The terminology used herein is for the purpose of describing particular embodiments only and is not intended to be limiting. The terms “comprises,” “comprising,” “including”, or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element. The terms “including” and/or “having,” as used herein, are defined as comprising (i.e., open language). The terms “a” or “an”, as used herein, are defined as one or more than one. The term “plurality,” as used herein, is defined as two or more than two. The term “another,” as used herein, is defined as at least a second or more. The description may use the terms “embodiment” or “embodiments,” which may each refer to one or more of the same or different embodiments.

When the terms “coupled”, “connected”, along with their derivatives, are used, these terms are not intended as synonyms for each other. For example, “connected” may be used to indicate that two or more elements are in direct physical or electrical contact with each other. “Coupled” may mean that two or more elements are in direct physical or electrical contact (e.g., directly coupled) or that two or more elements are not in direct contact with each other but yet still cooperate or interact with each other (e.g., indirectly coupled).

For the purposes of the description, a phrase in the form “A/B” or in the form “A and/or B” or in the form “at least one of A and B” means (A), (B), or (A and B), where A and B are variables indicating a particular object or attribute. When used, this phrase is intended to and is hereby defined as a choice of A or B or both A and B, which is similar to the phrase “and/or”. Where more than two variables are present in such a phrase, this phrase is hereby defined as including only one of the variables, any one of the variables, any combination of any of the variables, and all of the variables, for example, a phrase in the form “at least one of A, B, and C” means (A), (B), (C), (A and B), (A and C), (B and C), or (A, B and C).

Relational terms such as first and second, top and bottom, and the like may be used solely to distinguish one entity or action from another entity or action without necessarily requiring or implying any actual such relationship or order between such entities or actions. The description may use perspective-based descriptions such as up/down, back/front, top/bottom, and proximal/distal. Such descriptions are merely used to facilitate the discussion and are not intended to restrict the application of disclosed embodiments. Various operations may be described as multiple discrete operations in turn, in a manner that may be helpful in understanding embodiments; however, the order of description should not be construed to imply that these operations are order dependent.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means, when comparing various parts to one another, that the parts being compared are equal to or are so close enough in dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

Various embodiments of the systems, apparatuses, and methods have been described, and in many of the different embodiments many features are similar. To avoid redundancy, repetitive description of these similar features may not be made in some circumstances. It shall be understood, however, that description of a first-appearing feature applies to the later described similar feature and each respective description, therefore, is to be incorporated therein without such repetition.

From the foregoing, it will be appreciated that specific embodiments of the disclosure have been described herein for purposes of illustration, but that various modifications may be made without deviating from the scope of the disclosure. Accordingly, the disclosure is not limited except as by the appended claims.

What is claimed is:

1. A supplemental valve apparatus for implantation into the right atrium, comprising:
a supplemental valve; and
the supplemental valve coupled to at least one supplemental valve structure, the supplemental valve structure extends into at least one of the inferior vena cava or superior vena cava; and
the supplemental valve being positioned in or adjacent the right atrium.

2. The supplemental valve apparatus of claim 1, and wherein:
the supplemental valve is a biologic valve.

3. The supplemental valve apparatus of claim 1, and wherein:
the supplemental valve apparatus is positioned within the right atrium, and anchored in position by a structure extending into the inferior vena cava and into the superior vena cava.

4. The supplemental valve apparatus of claim 1, and wherein:
the supplemental valve apparatus includes one or more of a jacket and/or seal(s) to direct blood flow from the inferior vena cava and superior vena cava through the supplemental valve.

5. The supplemental valve apparatus of claim 1, and wherein:
the supplemental valve apparatus is configured to enable percutaneous delivery via a delivery system.

6. The supplemental valve apparatus of claim 1, and wherein:
the supplemental valve apparatus includes a delivery system for deploying a supplemental valve into the right atrium, comprising:
an inner shaft;
an outer shaft; and
a supplemental valve apparatus positioned between the inner shaft and the outer shaft, the outer shaft configured to be retracted with respect to the inner shaft to deploy the supplemental valve apparatus;
the supplemental valve apparatus having a supplemental valve, an inferior vena cava leg for anchoring at least a portion of the supplemental valve apparatus in the inferior vena cava, and a superior vena cava leg, for anchoring at least a portion of the supplemental valve apparatus in the superior vena cava.

7. The supplemental valve apparatus of claim 1, and wherein:
the supplemental valve apparatus includes a delivery system for deploying a supplemental valve into the right atrium, comprising:
a distal shaft;
a proximal shaft; and
a supplemental valve apparatus positioned between the distal shaft and the proximal shaft, the distal shaft configured to be advanced with respect to the proximal shaft to deploy the supplemental valve apparatus;
the supplemental valve apparatus having a supplemental valve, an inferior vena cava leg for anchoring at least a portion of the supplemental valve apparatus in the inferior vena cava, and a superior vena cava leg, for anchoring at least a portion of the supplemental valve apparatus in the superior vena cava.

8. The supplemental valve apparatus of claim 1, and wherein:
the supplemental valve apparatus includes a delivery system for deploying a supplemental valve into the right atrium, comprising:
a distal shaft;
a proximal shaft;
an outer shaft; and
a supplemental valve apparatus positioned between at least a portion of the distal shaft inner member and the outer shaft, the outer shaft configured to be retracted with respect to the distal shaft inner member to deploy at least a portion of the supplemental valve apparatus; the distal shaft configured to be advanced with respect to the distal shaft inner member to deploy at least a portion of the supplemental valve apparatus;
the supplemental valve apparatus having a supplemental valve, and at least one of an inferior vena cava leg for anchoring at least a portion of the supplemental valve apparatus in the inferior vena cava, and a superior vena cava leg, for anchoring at least a portion of the supplemental valve apparatus in the superior vena cava.

9. A method for treating a compromised tricuspid valve, comprising:
a supplemental valve apparatus including a supplemental valve;
leaving the existing tricuspid valve in position;
placing a supplemental valve in or adjacent the right atrium;
maintaining the position of the supplemental valve with a structure located at least in part within the superior vena cava and/or the inferior vena cava; and
directing blood flow from the superior vena cava and inferior vena cava through the supplemental valve.

10. The method of claim 9 wherein the supplemental valve apparatus is delivered to the desired location percutaneously.

11. The method of claim 9, and wherein:
the supplemental valve apparatus is deployed at least in part by retracting an outer shaft.

12. The method of claim 9, and wherein:
the supplemental valve apparatus is deployed at least in part by advancing a distal shaft.

13. The method of claim 9, and wherein:
the supplemental valve apparatus is deployed at least in part by advancing a distal shaft and retracting an outer shaft.

14. The method of claim 9, and wherein:
the supplemental valve is deployed in or adjacent the right atrium, and then the supplemental valve apparatus is anchored in position.
